(19) United States
(12) Patent Application Publication
(10) Pub. No.: US 2014/0356900 A1
(43) Pub. Date: Dec. 4, 2014

(54) Detection of luminal urinary catheter colonization

(71) Applicant: Empire Technology Development LLC, Wilmington (DE)

(72) Inventor: George Charles Peppou, Redfern (AU)

(73) Assignee: Empire Technology Development LLC, Wilmington, DE (US)

(21) Appl. No.: 14/110,151

(22) PCT Filed: May 31, 2013
(86) PCT No.: PCT/US13/43552

§ 371 Date: Oct. 6, 2013

(51) Int. Cl. C12Q 1/04; A61M 25/00; A61F 2/82

(52) U.S. Cl. CPC C12Q 1/04; A61F 2/82; A61M 25/0017; A61M 25/0009; A61M 25/0045; A61M 2025/0056; A61F 2310/00389
USPC 435/34; 604/111; 623/23.66; 604/544; 427/2.3

(57) ABSTRACT

Technologies are generally described for indwelling medical devices for detection of luminal catheter colonization. A substrate for use as an indwelling medical device includes an elevated biofilm releasing medium including a dye for release into urine on the occurrence of an elevated biofilm build-up on the substrate in the urine; and an elevated pH releasing medium including a dye on the substrate different from the dye used to detect biofilm build-up for release into urine on the occurrence of an elevated pH level in the urine. Methods of manufacture and use of the disclosed indwelling medical devices are also described.

### TECHNICAL FIELD

This disclosure relates generally to medical devices, and more particularly to indwelling medical devices for detection of luminal catheter colonization.

### BACKGROUND

Colonization of indwelling devices in the urinary systems, such as urinary catheters, is not inherently dangerous due to limited systematic risk of urinary tract infection (UTI). However, if urinary catheters are left in place for long periods occlusion of the device can occur due to the crystallization of calcium and magnesium phosphates from the urine. Ultimately, this may lead to complete blockage. If an occluded catheter is not replaced urinary retention and ultimately reflux of infected urine into the kidneys may occur, leading to pyelonephritis and septicaemia.

Treatment of catheterized patients with UTIs is also compromised, with antibiotic therapy less effective against infections caused by colonized catheters. This may prolong health issues and increase treatment costs. In many countries, like the U.S., hospitals are directly responsible for these costs of treating catheter acquired UTIs. Determining the presence of a biofilm within the catheter before antibiotic therapy is initiated may reduce overall cost by increasing likelihood of effective therapy, reducing treatment cost.

Unless otherwise indicated herein, the materials described in this section are not prior art to the claims in this application and are not admitted to be prior art by inclusion in this section.

### SUMMARY

Technologies are generally described for indwelling medical devices for detection of luminal catheter colonization. A substrate for use as an indwelling medical device includes an elevated biofilm releasing medium including a dye for release into urine on the occurrence of an elevated biofilm build-up on the substrate in the urine; and an elevated pH (i.e., an increase in the pH number and associated pH level which is the measure of the acidity of a solution and is calculated as the negative log of the concentration of hydrogen ions in the solution) releasing medium including a dye on the substrate different from the dye used to detect biofilm build-up for release into urine on the occurrence of an elevated pH level in the urine. Methods of manufacture and use of the disclosed indwelling medical devices are also described.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIGS. 1, 2 are perspective views illustrating an indwelling medical device in female and male use, respectively, according to this disclosure

FIG. 3 is a perspective view illustrating an indwelling medical device for contacting urine according to an illustrative example of this disclosure;

FIG. 4 is a perspective view illustrating an indwelling medical device for contacting urine according to an illustrative example of this disclosure;

FIG. 5 is a cross-sectional view taken along phantom line A-A of FIG. 4 illustrating the indwelling medical device contacting urine according to one illustrative embodiment of this disclosure;

FIG. 6 is a cross-sectional view taken along phantom line A-A of FIG. 4 illustrating the indwelling medical device contacting urine according to an alternative illustrative embodiment of this disclosure;

FIG. 7 is a perspective view illustrating an indwelling medical device for contacting urine according to this disclosure;

FIG. 8 is a cross-sectional view taken along phantom line A-A of FIG. 7 illustrating the indwelling medical device contacting urine according to one illustrative embodiment of this disclosure;

FIG. 9 is a flow diagram illustrating an example process of using a device according to this disclosure;

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

This disclosure is generally drawn, inter alia, to methods, apparatus, systems, devices, and computer program products related to detection of luminal catheter colonization.

Briefly stated, technologies are generally described for indwelling medical devices for detection of luminal catheter colonization. A substrate for use as an indwelling medical device includes an elevated biofilm releasing medium including a dye for release into urine on the occurrence of an elevated biofilm build-up on the substrate in the urine; and an elevated pH (i.e., an increase in the pH number and associated pH level which is the measure of the acidity of a solution and is calculated as the negative log of the concentration of hydrogen ions in the solution) releasing medium including a dye on the substrate different from the dye used to detect biofilm build-up for release into urine on the occurrence of an elevated pH level in the urine. Methods of manufacture and use of the disclosed indwelling medical devices are also described.

In describing more fully this disclosure, we make reference to the accompanying drawings, in which illustrative embodiments of the present disclosure are shown. This disclosure may, however, be embodied in a variety of different forms and should not be construed as so limited.

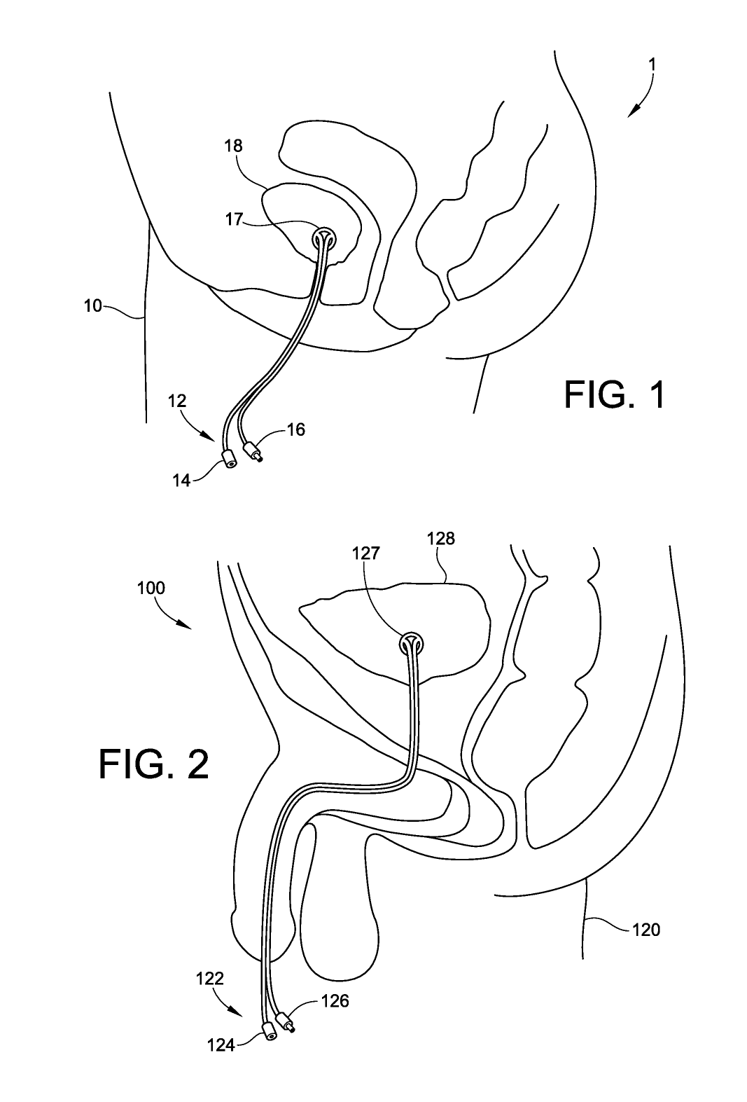

FIGS. 1 and 2 respectively depict examples of female and male patients 1, 100 into which a urinary Foley catheter 12, 122 has been inserted. The catheters 12, 122 include a first portion 14, 124 for drainage of urine and a second portion 16, 126 for inflation of the retaining portion 17, 127 of the Foley catheter within the patient's urinary bladder 18, 128.

Biofilm formation in indwelling devices, such as the urinary catheters illustrated in FIGS. 1 and 2, are well established. Due to the level of exposure of the region, large amounts of water present and nutrient availability the biofilm formation is common. Consequently infection risk is very high, with many patients receiving long-term catheterization receiving a UTI.

Measures to prevent colonisation including silver coatings and antibiotic impregnated materials have been shown to be ineffective in preventing biofilm formation. Hence, enhanced approaches to prevention must be sought, due to the high health care costs, particularly in the US, associated with treatment of catheter-acquired infections.

Occlusion of the catheter lumen is a concerning consequence of colonization of the urinary catheter. Bacteria capable of producing urease convert the abundant urea into ammonia raising the pH. Species are capable of raising the local pH to above 8.0. At this high pH calcium and magnesium phosphate in the urine crystallize on the wall of the catheter. This process is rapid and can occlude the catheter in as little as 40 hours. Consequences of occlusion are severe, as the catheter is contaminated by several species of bacteria whilst simultaneously being occluded. Infection is inevitable under these conditions; whilst urinary infections may be fatal this is a rare outcome. The occlusion instigates urinary retention, if the catheter remains in place this can lead to reflux of infected urine into the kidneys to occur, resulting in pyelonephritis and septicaemia. If this occurs the patient is likely to spend a greater amount of time in hospital increasing overall healthcare cost.

Having thus introduced background on urinary catheters, we now turn to features that are provided by this disclosure.

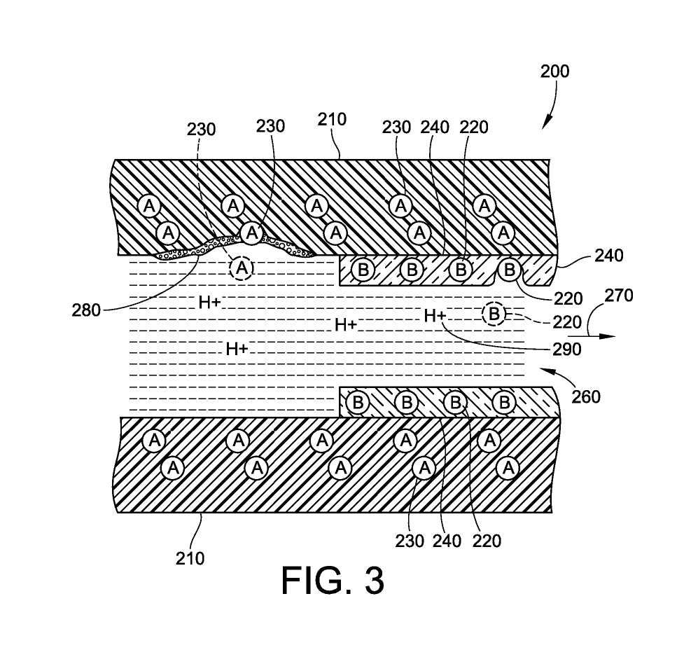

FIG. 3 is a perspective view illustrating a medical device 200 that may be inserted or implanted into a subject for contacting urine 260 according to an illustrative example of this disclosure. The urine is shown flowing from left to right in FIG. 3 as denoted by arrow 270 illustrated in FIG. 3. The medical device 200 is illustratively shown as a catheter 210 in this drawing but can be any insertable or implantable medical device. The catheter may be made from any material that is biomedically friendly to the body. The catheter 210 is illustratively provided with a coating 240 in this example.

As is known in the art, over time, a biofilm shown as biofilm 280 in FIG. 3. may form on one or more surfaces of materials inserted or implanted into a subject such as the catheter 210 in FIG. 3. The biofilm 280 may form from an aggregation of microorganisms on the surface. These biofilms may digestively feed on the surface where they form; causing that surface of the inserted or implanted material, such as the illustrated catheter, to structurally degrade or otherwise erode. This is shown in FIG. 3 by the recessed portion of the catheter 210 lying underneath the biofilm.

According to this disclosure, a dye 230 is incorporated into the catheter 210. When the catheter 210 has been eroded by biofilm colonization as illustrated by the recessed portion of the catheter 210 underneath the biofilm 280, the dye 230 that resides in the eroded portion of the catheter is released into the urine. The dye 230 that is shown in FIG. 3 in phantom illustrates a dye 230 that has been released into the urine due to biofilm erosion. The released dye 230 is carried with the urine and may be detected by a caregiver such as by detection of the dye in urine that has been excreted.

As also disclosed, a dye 220 is illustratively incorporated into the coating 240 on the catheter 210. The coating 240 is designed to release the dye 230 in the presence of an elevated pH level. Details of how the coating 240 may be so designed are provided in detail later in this disclosure. As previously described, an elevated pH level is an increase in the pH number and associated pH level which is the measure of the acidity of a solution and is calculated as the negative log of the concentration of hydrogen ions in the solution.

These hydrogen ions are identified as hydrogen ions 290 in FIG. 3. When the coating 240 is in the presence of an elevated pH level, the coating 240 releases the dye 220 into the urine. FIG. 3 illustrates this release by the recessed portion of the coating which allows the dye 220 to be released from the coating. The dye 220 that is shown in FIG. 3 in phantom illustrates a dye 220 that has been released into the urine due to an elevated pH level in the urine. The released dye 220 is carried with the urine and may be detected by a caregiver such as by detection of the dye in urine that has been excreted.

In the example shown in FIG. 3, the dye 230 is described as being incorporated into the catheter 210 and the dye 220 is described as being incorporated into the coating 240 on the catheter 210. In this example, the catheter provides a substrate, that is to say, a structure for holding the dye 230 and the coating provides a substrate (i.e., structure) for holding the dye 220. In alternative embodiments described later in this disclosure, the dye 220 may be incorporated into the substrate (i.e., structure) formed by the catheter and the dye 230 may be incorporated into the substrate (i.e., structure) provided by the coating. In still other examples, both dyes may be incorporated into the substrate (i.e., structure) formed by the catheter and/or provided by the coating. Where the material inserted or implanted is not provided with a coating, both dyes may be incorporated into the substrate (i.e., structure) formed by the inserted or implanted material. In addition, where the material inserted or implanted is provided with an accessory as described below, one or both dyes may be incorporated into either or both the substrate (i.e., structure) formed by the inserted or implanted material and the substrate (i.e., structure) formed by the accessory. If a coating is provided onto either or both inserted or implanted medical device and accessory, the dyes may be incorporated into one or more of the structural components of the inserted or implanted material, the accessory, and coating that in combination may be inserted or implanted into a subject in the latter example. These and other substrates (i.e., structures) which may be provided by or in combination with a material for insertion or implantation into a subject will be apparent to one skilled in the art in view of this disclosure.

Illustratively, each of the dye 230 used to detect biofilm build-up and the dye 220 used to detect elevated pH levels are of a different color so that that the caregiver may determine which of the conditions of infection or occlusion is triggering a color change in the urine. With the presence of either or both dyes in the urine, this disclosure thus provides a caregiver with a valuable tool for use in treating a subject.

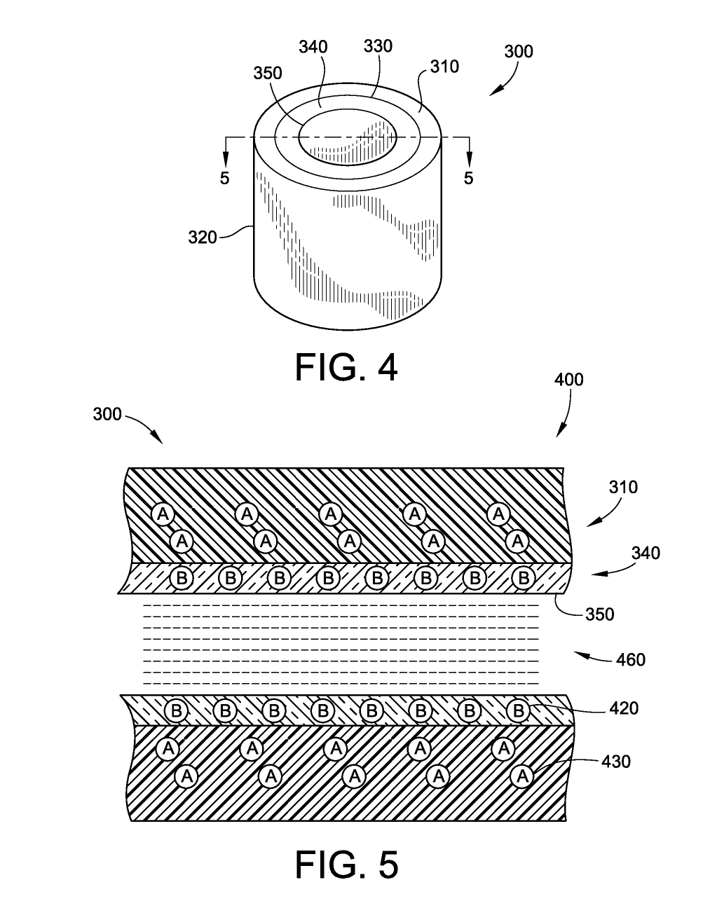

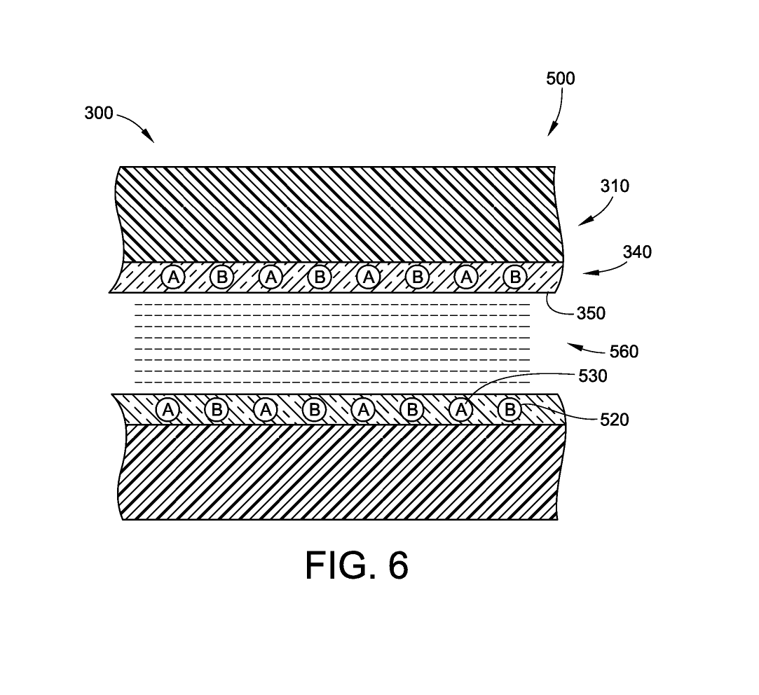

FIG. 4 is a perspective view illustrating an indwelling medical device 300 for contacting urine (not shown) according to an illustrative example of this disclosure. FIG. 5 is a cross-sectional view taken along phantom line A-A of FIG. 4 illustrating the indwelling medical device 300 contacting urine 460 according to one illustrative embodiment of this disclosure.

The indwelling medical device 400 shown in FIGS. 4 and 5 includes a substrate 300 configured to contact urine 460. In this example, the substrate 300 includes material 310 for inserting or implanting into a subject and a coating 340 on the material 310. The material 310 and the coating 340 are described in greater detail below. A first detecting material 420 is disposed with the substrate configured to be released into the urine on the occurrence of an elevated pH level in the urine. A second detecting material 430 is disposed with the substrate configured to be released into the urine on the occurrence of an elevated biofilm on the substrate. A detection of the first detecting material in the urine indicates an elevated pH level in the urine. A detection of the second detecting material in the urine may indicate an elevated biofilm on the substrate. The occurrence of an elevated pH level may indicate a condition conducive to an occlusion of the device and the occurrence of an elevated biofilm on the substance may indicate a condition conducive to an infection. Hence, this disclosure enables the detection of both occlusion and infection through an integrated solution. The two stage response provided by this disclosure, during colonization and during crystallization, provide these and other advantages.

More specifically, in FIG. 5, substrate 300 may illustratively be a catheter 310 coated with a hydrogel 340. However, the substrate of this disclosure is not limited to a two part substrate (i.e., structure) of catheter and a coating. The substrate (i.e., structure) may be a catheter or other insertable or implantable medical device alone. The insertable or implantable medical device substrate (i.e., structure) may be provided with an accessory component or other structural component in which case the substrate of this disclosure may be provided by the insertable medical device alone, by the accessory or other structural component, by a coating provided on the insertable or implantable medical device or the accessory or other structural component, or by any combination of these pieces of substrates (i.e., structures). These and other configurations for use as a substrate (i.e., structure) according to this disclosure will be apparent to one skilled in the art from this disclosure.

In the FIG. 5 example, the first detecting material 420 is disposed in the catheter and the second detecting material 430 is disposed in the hydrogel. In particular, the second detecting material is added to the material used to make the catheter such that the material of the catheter encases the second detecting material. The first detecting material is added to the hydrogel. In this example, the catheter is configured to release the second detecting material into the urine on the detection of an elevated biofilm growth on the catheter. For example, plasticizers used in PVC, a material commonly used to manufacture urinary catheters, have been shown to provide nutrition to the biofilm as the plasticizers diffuse from the polymer matrix. Over time the material swells and is degraded further. While many materials are susceptible to biodegradation and are useable according to this disclosure, PVC is already commonly used to produce medical devices. In addition, the hydrogel 340 is configured to release the first detecting material into the urine on the detection of an elevated pH level in the urine.

More specifically, the catheter releases the second detecting material from the catheter as the biofilm erodes the walls of the catheter. This release may occur by degradation of the encapsulating material, diffusion, or in other ways well known to those skilled in the art. In addition, the hydrogel 340 is designed to swell at the elevated pH level associated with occlusion, thereby releasing the first detecting material residing in the hydrogel 340 into the urine to signal the onset of occlusion as described in greater detail below.

Unlike biofilms forming on other medical devices urease producing microbes increase the pH of their environment. Hence, biofilm growth in the urease producing environment increases the pH level of the urine which is detected by the second detecting material according to this disclosure. Also, the elevated pH level associated with biofilm growth is different from the elevated pH level associated with the onset of occlusion. This disclosure advantageously provides an encapsulating material for the second detecting material that releases its detecting material payload at a different pH level that is lower than the level of pH required to release the detecting material payload enclosing the first detecting material.

Normal urine pH varies. Typically urine ranges from pH 4.8-7.4 with average pH approximately 7.0, with raised pH generally a side effect of medications while lowered pH is predominantly a consequence of diet. Hence, the release mechanism for the second detecting material (e.g., biofilm detecting dye) must be stable under mildly acid and neutral conditions and release dye above neutral pH to indicate biofilm growth. Acid sensitive encapsulants are typically not suitable where the encapsulants in the indwelling medical device are in contact with urine. Illustratively, the pH level for releasing the second detection material may be at a pH level between about 7 and 7.4. The release mechanism of the first detecting material must be sensitive to the high pH generated by the urease producing bacteria. Under these conditions (ph>7.4) release mechanism for the first detecting material may release the first detecting material (e.g., occlusion indicating dye). The pH level for releasing the second detection material is illustratively substantially at or above 7.4, substantially between about 7.4 and 8 pH values, substantially between about 7.25 and 7.74 pH; and most preferably, substantially at or about 7.4 pH.

FIG. 5 shows the hydrogel 340 overlaying the catheter; thus requiring biofilm to erode the hydrogel 340 layer in order to reach the surface of the catheter. In an alternative embodiment, the hydrogel 340 may be disposed on certain one or more surfaces of the catheter, leaving other surfaces of the catheter that have not been provided with the hydrogel 340 layer to be directly exposed to urine for direct biofilm growth. In another embodiment, the properties of the hydrogel may be designed to allow microbes to pass through the hydrogel to contact the surface of the catheter to allow for direct biofilm growth on the catheter for detection according to this disclosure.

The indwelling medical device shown in FIG. 5 is illustratively a Foley catheter. In other illustrative embodiments, the indwelling medical device may be a guidewire, a sheath. Alternatively, the indwelling medical device may be any medical device that may be inserted or implanted into a subject. Nonlimiting examples of indwelling medical devices include balloon catheters, biomedical implants, condom catheters, double-channel catheters, elbowed catheters, electrode catheters, endotracheal tubes, female catheters, fluid-filled catheters, Foley catheters, Gouley catheters, indwelling catheters, intrauterine devices, needleless connectors, peritoneal dialysis catheters, prostatic catheters, prosthetic joints, self-retaining catheters, snare catheters, stents, Tenckhoff catheters, toposcopic catheters, two-way catheters, ureteral catheters, winged catheters.

As used herein, catheter 310 may be a medical device that is inserted into a cavity of the body typically to withdraw or introduce fluid. The catheter typically includes a shaft which may contain one or more lumens. The catheter may be inserted into a subject for introduction of fluids, for removal of fluids, or both. The subject may be a vertebrate subject such as a mammalian subject. Examples of mammalian subjects include a human, a dog, a cat, a horse, etc. Catheters may be soft catheters which are thin and flexible or may be provided in varying levels of stiffness depending on the application. Catheters may be inserted in the body to treat diseases or perform a surgical procedure. A catheter may be an indwelling catheter left inside the body, either temporarily or permanently as a permcath. By modifying the material or adjusting the way catheters are manufactured, catheters may be tailored for a wide range of medical uses including cardiovascular, urological, gastrointestinal, neurovascular, ophthalmic, and other medical applications. Some commonly used catheters include peripheral venous catheters, which may be inserted into a peripheral vein, usually in the hand or arm, for the administration of drugs, fluids, and so on. Some other illustrative examples of catheters have been previously described.

As used herein, catheter 310 may include various accessory components, subassemblies, or other accessory parts. For instance, the catheter may include molded components, over-molded components, subassemblies, or other accessory components or parts. The catheter may also include connecting fittings such as hubs, extension tubes, and so on. Various catheter tips designs are known. These designs include stepped tips, tapered tips, over-molded tips and split tips for multilumen catheters, and so on.

In an alternative illustrative embodiment, the indwelling medical device may illustratively be a ureteral stent. As used herein, a stent is a mold or a device of suitable material used to provide support for structures for holding one or more biomaterials or biostructures in place. These biomaterials and biostructures may include skin, arteries, bodily orifice or cavity, or other biomaterial or biostructure of the body of the subject into which the stent may be placed. Illustrative stents may include urethral, ureteral stents, and so on. Stents may be used to treat urinary tract. Stents may also be used to treat other medical conditions. The stents may be of any shape or configuration. The stents may include a hollow tubular structure, which may be useful in providing flow or drainage through ureteral, biliary, or other lumens. Stents may be coiled or patterned as a braided or woven open network of fibers, filaments, and so on. Stents may also include an interconnecting open network of articulable or other segments. Stents may have a continuous wall structure or a discontinuous open network wall structure.

As used herein, a stent may include a stent cover which may include a tubular or sheath-like structure adapted to be placed over a stent. The stent cover may include an open mesh of knitted, woven or braided design. The stent may be made of any material useful for providing structure for holding one or more biomaterials or biostructures in place. These materials may include metallic and non-metallic materials. They may also include shape memory materials. Metallic materials may include shape memory alloys such as nickel-titanium alloys. They may also include other metallic materials such as stainless steel, tantalum, nickel-chrome, cobalt-chromium, and so on.

Illustratively, material for making the catheter may be a polyvinyl chloride generally referred to by the abbreviation PVC. Alternatively, materials may illustratively be selected from various grades of biocompatible materials including plasticizers, silicones and latex rubbers. As used herein, the term “biocompatible” means a material that is not substantially toxic to the human body and that does not significantly induce inflammation or other adverse responses in body tissues.

The shaft of the catheter may be made using techniques commonly known in the catheter art. For example, the shaft may be formed by extrusion, such as by a thermoplastic extrusion or a thermoset extrusion as is well known in the catheter art. A coating process such as solvent casting may also be used to form the shaft.

In FIG. 5, hydrogel 340 is illustratively a Carboxymethylchitosan hydrogel. Carboxymethylchitosan hydrogel has a swelling behavior of the polymer that is pH sensitive due to the presence of the chitosan acid moieties. In this embodiment, the greatest swelling generally occurs at a pH value of substantially around 7.4. Alternatively, any hydrogel having a swelling behavior of the polymer that is pH sensitive due to the presence of acid moieties such as to experience swelling between pH value substantially at or above 7.4 pH, substantially between about 7.4 and 8 pH values, or substantially between about 7.25 and 7.74 pH may be used with this disclosure. Most preferably, any hydrogel having a swelling behavior of the polymer that is pH sensitive due to the presence of acid moieties such as to experience swelling between pH value substantially at or about 7.4 pH values may be used with this disclosure. The hydrogel is illustratively coated onto the catheter.

The first and second detecting materials are illustratively any non-toxic dye. As used herein, “non-toxic” is any dye that is not injurious to health. Both dyes may be selected from any of the following list of dyes. The dyes must be selected such that the first and second dye are different colors in aqueous solution. The below list includes a small number of examples, in reality hundreds of dyes could be selected for application to this disclosure. The list of dyes for use as first and second detecting materials includes Annatto, Chlorophyllin, Cochineal, Betanin, Curcumin, Lycopene, Yellow No. 5, β-carotene, rifampin, Yellow No. 6, tetracycline, Red No. 40, Red No. 3, Blue No. 2, Evan's Blue, Green No 3, Blue No. 1, methylene blue, indocyanine green, Riboflavin, Tartrazine, Quinoline yellow, Cochineal, Carminic acid, Azorubine, Amaranth, Cochineal Red, Erythrosine, Allura Red AC, Patent Blue V, lndigotine, Brilliant Blue FCF, Green S, Brilliant Black BN, Vegetable carbon, Capsanthian, Beta-apo-8′-carotenal, Lycopene, Ethyl ester of beta-apo-8′-carotenoic acid, Lutein, Canthaxanthin, Anthocyanins.

As described below, the dyes may be carried from the catheter surface to the urine collection vessel, consequently they do not enter the body in any significant quantity, meaning biocompatibility of the dye is not essential. Consequently a range of natural and synthetic dyes which do not have no demonstrated toxicity are suitable for this application.

The first detecting material 420 is illustratively disposed in the hydrogel 340 with the first detecting material configured to be released into the urine on the occurrence of an elevated pH level in the urine. In the illustrative embodiment using Carboxymethylchitosan hydrogel, the Carboxymethylchitosan hydrogel has a swelling behavior of the polymer that is pH sensitive with greatest swelling occurring at a pH value of substantially around 7.4. Hence, at pH in the urine of substantially around 7.4 pH, the Carboxymethylchitosan hydrogel swells causing the dye to be released into the urine. The detection of the first detecting material indicates an elevated pH level in the urine. In embodiments in which the first detecting material is disposed in a hydrogel, a hydrogel having a swelling behavior of the polymer that is pH sensitive substantially at or above 7.4, substantially between about 7.4 and 8 pH values, substantially between about 7.25 and 7.74 pH; and most preferably, substantially at or about 7.4 pH values may be used with this disclosure. The diffusion of the first detecting material out of the hydrogel may be controlled to the desired pH range by adjusting the hydrogel composition so that the dye being released is less soluble or more mobile within the hydrogel. In addition, diffusion may be controlled by selection of dye based on molecular weight. The larger the molecular size, the lower will be the diffusion rate through the hydrogel, given comparable solubility. These parameters of hydrogel composition adjustment and selection of molecular weight of an agent for release from a hydrogel are well known to those skilled in the art and may be readily applied to adjust the release of the dye as described in this disclosure to provide the desired result. Other parameters for controlling the release of the first detecting material are also well known in the art.

The second detecting material 430 is illustratively disposed in the catheter with the dye configured to be released into the urine on the occurrence of an elevated biofilm on the substrate. In the illustrative embodiment, the material used for the catheter is PVC and the second detecting material is disposed in the PVC. In alternative illustrative embodiments, materials used for the catheter may illustratively be selected from various grades of biocompatible materials and the second detecting material is disposed in the biocompatible material. In such PVC or other biocompatible material, the second detecting material may diffuse to some extent through the polymer matrix toward an external surface and/or the physiological fluid, such as the urine with which the second detecting material may be in contact may diffuse into the polymeric matrix. The dye then dissolves in the physiological fluid. A concentration gradient is believed to be set up at or near the matrix region, and the dye in solution is then released via diffusion into the surrounding physiological fluid and local tissues. Detection of the second detecting material in the urine indicates an elevated biofilm on the substrate.

Current urinary catheters incorporate a hydrogel coating to provide improved surface properties, consequently overall device properties will not be alter significantly. The hydrogel may be applied to only the lumen of the catheter or to the entire device surface. Illustratively, the first detecting material and the second detecting material are disposed along an inside wall of at least one of the one or more lumens of the catheter. Alternatively, the first detecting material and the second detecting material may be disposed along any surface of the catheter including an outside wall of the one or more lumens of the catheter or on any surface of an accessory component, subassembly, or other accessory part that may be used with the catheter that allows the first detecting material and the second material to be released into the urine either directly or indirectly.

In an illustrative operation, the dye encapsulated in the PVC or other biomaterial forming the catheter, in the presence of a biofilm, some one of the encapsulants containing the second detecting material (e.g., dye) is degraded by the altered pH generated by the biofilm as it grows. The dye is released and dissolves into the urine, causing a color change in the collection vessel indicating early stage device colonization. At this stage, due to the limited risk of urinary infections the device may remain in place unless antibiotic therapy is initiated.

As the biofilm grows it may begin to cause the crystallization of magnesium phosphates and calcium from the urine, eventually causing the occlusion of the device potentially leading to serious complications including septicaemia. Under conditions favoring crystallization occurring the second detecting material (e.g., dye) is released indicating the device must be replaced to prevent complications.

The dyes may be carried from the catheter surface to the urine collection vessel, consequently they do not enter the body in any significant quantity, meaning biocompatibility of the dye is not essential. Consequently a range of natural and synthetic dyes which do not have no demonstrated toxicity are suitable for this application.

The usage of device does not require any significant alterations to current practice. Insertion and general usage of the device are unchanged. As with current practice urine output will be monitoring at regular intervals and through this device status can be determined. As the device has two different colored dyes to indicate two states of the device (colonized and crystalline biofilm formation), the disclosure allows for the detection of both biofilm colonization and occlusion using an integrated solution.

The presence of the dye used to detect biofilm build-up does not require immediate action as colonization of the device is often not clinically significant in the absence of a symptomatic UTI. Consequently detection of dye used to detect biofilm build-up in output urine does not require any immediate action, instead the result must be recorded to ensure if antibiotic therapy is initiated it will be effective. In the case of development of a symptomatic infection this previous record is checked. In the case of colonization the device is removed and replaced before antibiotic therapy is initiated.

The detection of the dye used to detect elevated pH levels requires a more rapid response. Once the conditions are met for crystallization to occur full occlusion of the lumen can occur within 24 hours. Consequently detection of this dye requires the catheter to be changed quickly to prevent pyelonephritis and septicaemia.

### Example

Particles of methylene blue are formed using the vibration nozzle method, a solution of the dye and gelling agent (e.g. 3% (w/v) alginate) are passed through the nozzle to generate particles. Particles of a uniform size (which may be set from 20-10,000 micron, though a size of approximately 50-100 micron is preferred for application in this example) are generated and immediately immersed in a solution to provide gelatin, in the case of alginate, a 0.2M calcium chloride solution. The particles have a high concentration of the methylene blue, in excess of 10% (w/v).

The dye containing particles are mixed with PVC at a ratio of approximately 5% (w/w). The PVC is then processed into the shape of the medical device using standard processing methods. A hydrogel as described below is then applied to the surface of the formed PVC device. The hydrogel provides both enhanced biocompatibility of the device and a mechanism for dye release in the presence of elevated pH, conditions necessary for encrustation to occur.

A Carboxymethylchitosan hydrogel is prepared containing the dye Betanin. The Carboxymethylchitosan was prepared in accordance with protocols known in the art, such as the development of pH sensitive hydrogel for intestinal deliver of meth prednisolone using chitosan derivative. Loading of dye into hydrogel is achieved by preparing a solution of Carboxymethylchitosan in 0.1N acetic acid and Betanin 10% (w/v), under stirring at 5000 rpm for 30 minutes. Carbopol 934 was prepared in 1.75 M acetic acid, the Carboxymethylchitosan is then gradually added to the carbopol solution. The solution is then spray coated onto the devices. The devices are then maintained at room temperature for 12 hours to facilitate cross-linking before being dried under vacuum. The devices may then be packaged and sterilized for use.

In this example, the release of the detecting material (e.g., the dye) is not an encapsulant. Hence, release of the dye may occur over a longer period. Increasing the quantity of dye in the material may assist in providing a strong signal. In addition, utilizing a hydrogel as a surface coating is reasonable as many current urinary catheters are coated in a hydrogel media to provide a hydrophilic surface in contact with the body. Consequently utilizing a hydrogel as a dye release mechanism may not require significant changes to current manufacturing practices.

In an alternative embodiment of this disclosure, FIG. 6 shows a cross-sectional view taken along phantom line A-A of FIG. 4 illustrating the indwelling medical device 300 contacting urine 560 according to an alternative illustrative embodiment of this disclosure. In FIG. 6, substrate 300 may illustratively comprise an insertable or implantable medical device such as a catheter 310 provided with a coating 340 and both the first detecting material 520 and the second detecting material 530 may be disposed in the coating.

In FIG. 6, a first detecting material 520 and a second material 530 may be disposed in a coating 340, which illustratively may be a hydrogel. In this illustrative example, the hydrogel 340 is configured to release the second detecting material into the urine as biofilm growth eats away at the hydrogel. More specifically, the second detecting material is illustratively encapsulated in a pH responsive material that releases its payload at a pH level that is lower than the pH level at which the hydrogel swells. The encapsulant may be a pH responsive nanoparticle, a pH responsive polyester urea, or other pH responsive biocompatible material. In the case of the nanoparticles, for example, the nanoparticles are impregnated into the hydrogel such that the hydrogel holds both the nanoparticle encapsulated second detecting material as well as the first detecting material in place. At the lower pH level associated with biofilm formation, the nanoparticles may be degraded, diffused, or otherwise chemically altered causing the second detecting material to be released into the urine. The hydrogel does not swell at this lower pH level; hence the first detecting material remains within the hydrogel. At the higher pH levels associated with the onset of occlusion, the hydrogel swells, causing the first detecting material to be released into the urine to signal the onset of occlusion. The swelling behavior of the hydrogel polymer may be pH sensitive substantially at or above 7.4 pH values. The swelling behavior of the polymer may be pH sensitive at substantially at or above 7.4, substantially between about 7.4 and 8 pH values, substantially between about 7.25 and 7.74 pH; and most preferably, substantially at or about 7.4 pH values.

In FIG. 6, the diffusion of the first detecting material out of the hydrogel may be controlled to the desired pH range by adjusting the hydrogel composition so that the dye being released for pH level detection is less soluble or more mobile within the hydrogel. The diffusion of the first detecting material and the second detecting material in the hydrogel coating may be further controlled by selection of detecting material (e.g., dye) based on molecular weight. The larger the molecular size, the lower will be the diffusion rate through the hydrogel, given comparable solubility. These parameters of hydrogel composition adjustment and selection of molecular weight of an agent for release from a hydrogel are well known to those skilled in the art and may be readily applied to adjust the release of the dye as described in this disclosure to provide the desired result. Other parameters for controlling the release of the detecting materials are also well known in the art.

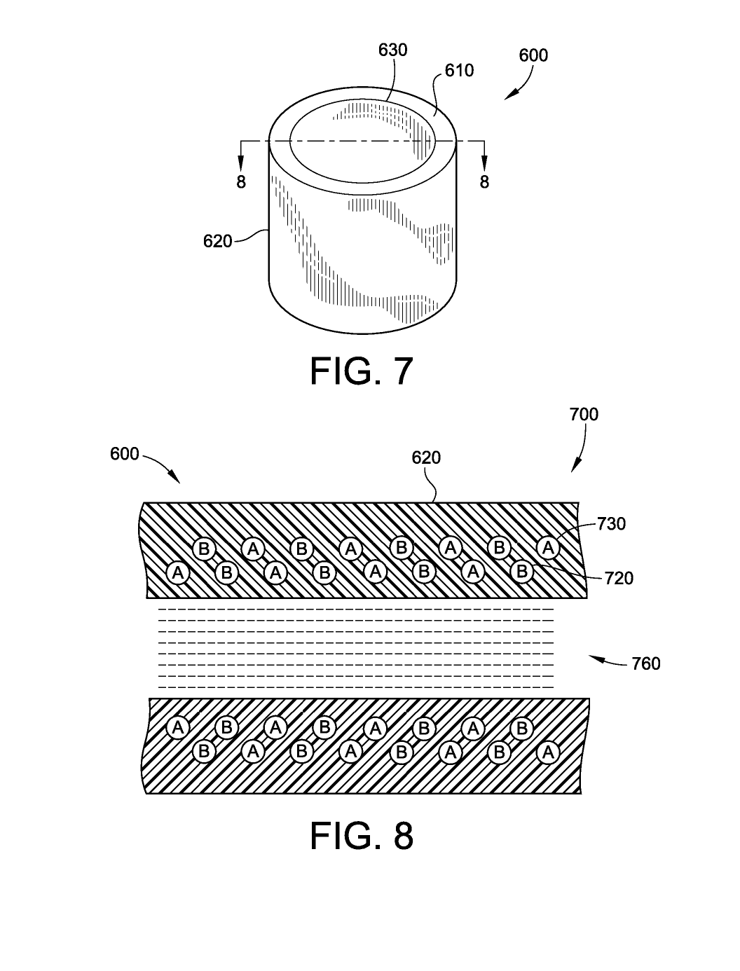

In yet another illustrative embodiment, FIG. 7 shows a perspective view illustrating an indwelling medical device 600 for contacting urine (not shown) according to this disclosure. FIG. 8 is a cross-sectional view 700 taken along phantom line A-A of FIG. 7 illustrating the indwelling medical device 700 contacting urine 760 according to one illustrative embodiment of this disclosure.

The indwelling medical device 600 shown in FIGS. 7 and 8 may include a substrate 600 configured to contact urine 760. A first detecting material 720 is disposed with the substrate configured to be released into the urine on the occurrence of an elevated pH level in the urine. A second detecting material 730 is disposed with the substrate configured to be released into the urine on the occurrence of an elevated biofilm on the substrate. A detection of the first detecting material in the urine indicates an elevated pH level in the urine. A detection of the second detecting material in the urine indicates an elevated biofilm on the substrate.

In FIG. 8, substrate 600 may illustratively comprise an insertable or implantable medical device, such as a catheter 620, and the first detecting material 720 may be disposed in the catheter and the second detecting material 730 may also be disposed in the catheter. In this illustrative embodiment, both the first detecting material 720 and the second detecting material 730 may be disposed in the catheter 620. Illustratively, the first detecting material may be encased in an encapsulant that is responsive to the higher pH level at which the detecting material is to be released into the urine. The second detecting material need not be encapsulated in this example. The encapsulated first detecting material and the second detecting material may be mixed with PVC illustratively used to make the catheter in the illustrative example. Advantageously, the encapsulating material used for the first detecting material in the PVC is selected to release its detecting material payload at a different pH level that is higher than the level of pH required to release the detecting material payload enclosing the second detecting material, which in this example is the PVC material used to make the catheter itself.

For example, a nanoparticle encapsulation of the first detecting material provides effective drug release at pH greater than 7.4. The nanospheres may be manufactured using PLGA and a pH sensitive methacrylate copolymer. Drug release may be demonstrated to be highly pH dependent. Application of nanospheres such as these to the disclosure enable the first detecting material to be encased and mixed with the PVC polymer during processing and manufacture or be attached to a surface coating, either adhered or applied in a surface hydrogel coating.

Alternatively the first detecting material may be encapsulated using polyester urea, a polymer susceptible to degradation by urease, the enzyme responsible for creating the high pH environment responsible for crystallization occurring. Polyester urea has seen limited application, it has been demonstrated as an effective scaffold for tissue engineering. Forms of polyester urea are capable of being melt processed, simplifying production of encapsulated particles. As previously discussed, in this example, the material used to form the catheter forms the encapsulating medium for the second detecting material. The polymer used to form the catheter provides a coating to the particles of encapsulated first detecting material (e.g., dye) and to the uncapsulated particles of the second detecting material (e.g., dye), allowing second detecting material (e.g., dye) release on coating degradation. Release of the first detecting material occurs on conditions of elevated pH levels as herein disclosed.

Hence, the encapsulating material used for the first detecting material in the PVC is selected to release its payload at a higher pH level than the material used to make the catheter that is encapsulating the second detecting material in this example. Illustratively, the pH level for the encapsulating material used for releasing the second detection material is selected to degrade, diffuse, or etc. at a pH level up to about 7. The pH level for the encapsulating material used for releasing the first detection material is selected to illustratively degrade, diffuse, or etc. substantially at or above 7.4, substantially between about 7.4 and 8 pH values, substantially between about 7.25 and 7.74 pH; and most preferably, substantially at or about 7.4 pH values. Hence, biofilm degradation, diffusion, etc. of the PVC layer will at pH levels below about 7.4 only degrade, diffuse, etc. the encapsulating material holding the second detecting material payload; causing the second detecting material to be released into the urine to indicate a potential condition of an infection.

Advantageously, the encapsulating material holding the first detecting material payload does not degrade, diffuse, etc. under conditions of lower pH. Hence, even if any encapsulating material holding the first detecting material payload is broken away from the PVC, due to erosion of the PVC by microbes forming the biofilm, and enters the urine, it does not release the first detecting material payload since the pH level is not at the higher pH level required to do that. When the pH level in the urine raises to that higher pH level, however, the material encapsulating the first detecting material degrades, diffuses, etc, to release its first detecting material into the urine; signaling a condition which may indicate the onset of occlusion. In this way and in other ways known to those skilled in the art, the PVC or other plasticizer may be used to hold the second detecting materials for release in response to metabolism of biofilm and to hold the encapsulated first detecting material which encapsulants release only under elevated pH levels that may indicate a condition an onset of occlusion.

In alternate embodiments, antimicrobial factors may also be applied to the substrate configured to be released into the urine with the release of the second detecting material. Alternatively, these factors may be applied to the substrate configured for release of either or both first detecting material and second detecting material. These antimicrobial factors may include drugs, chemicals, or other substances that either destroy microbes, prevent their development, or inhibit their pathogenic action. Antimicrobial factors may include antibacterial drugs, antiviral agents, antifungal agents, and antiparasitic drugs.

In alternate embodiments, antifouling factors may also be applied to the substrate configured to be released into the urine with the release of the second detecting material. Alternatively, these factors may be applied to the substrate configured for release of either or both first detecting material and second detecting material. These antifouling factors which may be released concurrently with the dye include, but are not limited to the follow classes: antibiotics, oxidizing agents (e.g. iodine, peroxide), alcohol, bacteriocins, chelating agents, NaCl, CaCl2, MgCl2, surfactants, urea and/or antimicrobial peptides (AMPs). Furthermore, the surfaces of the devices may be antimicrobial surfaces, which can be accomplished by embedding silver, copper, or the quaternary ammonium compound 3-(Trimethoxysilyl)-propyldimethyloctadecyl ammonium chloride (Si-QAC). Also, the surfaces' antimicrobial nature may be enhanced by modifying a physical factor such as surface smoothness or the hydrophobic nature to prevent adhesion.

In alternate embodiments, enzyme factors may also be applied to the substrate configured to be released into the urine with the release of the first detecting material. Alternatively, these factors may be applied to the substrate configured for release of either or both first detecting material and second detecting material. Enzyme factors known to disrupt biofilm during growth and maturation may include: cellulase, polysaccharide depolymerase, alginate lyase, disaggregatase, esterases, dispersin B, DNase I.

In alternate embodiments, factors to reduce pH may also be applied to the substrate configured to be released into the urine with the release of the first detecting material. Alternatively, these factors may be applied to the substrate configured for release of either or both first detecting material and second detecting material. Factors to reduce pH are well known to those skilled in the art.

In alternate embodiments, factors to reduce crystallization may also be applied to the substrate configured to be released into the urine with the release of the first detecting material. Alternatively, these factors may be applied to the substrate configured for release of either or both first detecting material and second detecting material. Factors to insolvate crystals may include polyphosphates or other agents known to solvate calcium pyrophosphate crystals

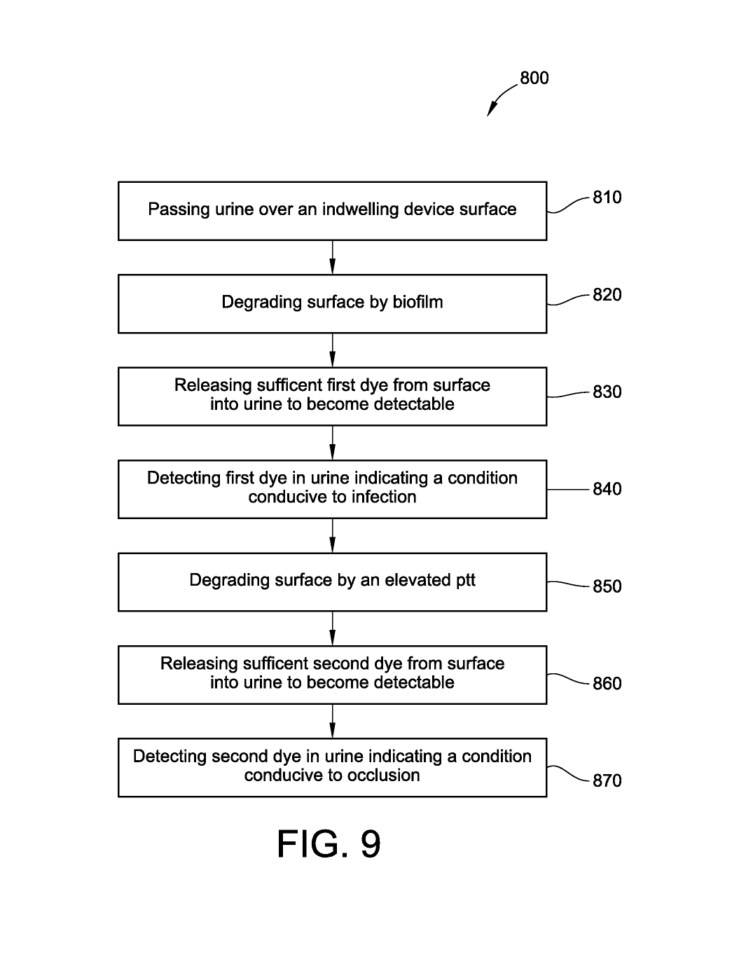

FIG. 9 is a flow diagram 800 illustrating an example process of using a device according to this disclosure. The process may be used for detecting occlusion and the growth of a biofilm on an insertable or implantable indwelling medical device. According to the process, urine is passed over a surface of the indwelling medical device 810. The surface of the indwelling medical device is degraded by a biofilm growing thereon 820 by an amount sufficient to release a first dye therefrom. A sufficient amount of the first dye is released from the surface of the indwelling medical device into the urine 830 to become detectable in urine. The first dye is detected in the urine 840 indicating a condition conducive to an infection. The surface of the indwelling medical device s degraded by an elevated pH 850 sufficient to release a second dye therefrom. A sufficient amount of the second dye is released from the surface of the indwelling medical device 860 into the urine to become detectable in urine. The second dye is detected in the urine 870 indicating a condition conducive to an occlusion of the indwelling device

A method for making a medical device may be as follows. A substrate is provided for use as an indwelling medical device. An elevated biofilm releasing medium including a first dye is applied to the substrate for release into urine on the occurrence of an elevated biofilm build-up on the substrate in the urine. An elevated pH releasing medium including a second dye is applied to the substrate different from the first dye for release into urine on the occurrence of an elevated pH level in the urine. The occurrence of an elevated pH level may indicate a condition conducive to an occlusion of the medical device. The elevated pH level for trigging release of the second dye from the elevated pH releasing medium may be at a pH level of substantially about 7.4; substantially between about 7.25 and 7.74 pH; substantially between about 7.4 and 8 pH values, or substantially at or about 7.4 pH. The occurrence of an elevated biofilm on the substance may indicate a condition conducive to an infection.

The method for making the medical device may include one or more of the following further steps. An antimicrobial factor may be applied to the substrate configured to be released into the urine with the release of the first dye An antifouling factor may be applied to the substrate configured to be released into the urine with the release of the first dye; applying an enzyme factor capable of damaging expolymer structure to the substrate configured to be released into the urine with the release of the second dye. A pH reducing factor may be applied to the substrate configured to be released into the urine with the release of the second dye; applying a calcium phyrophosphate crystal salvation factor to the substrate configured to be released into the urine with the release of the second dye; or other factors known to those skilled in the art. The antimicrobial factors, antifouling factors, enzyme factors, pH reducing factors, and phyrophosphate crystal salvation factors for use with this method are as previously disclosed.

In addition, the step of applying an antifouling factor on the substrate may include the step of applying an antimicrobial surface to the substrate. The step of applying an antimicrobial surface to the substrate may be performed by disposing with the substrate a silver, copper, or the quaternary ammonium compound 3-(Trimethoxysilyl)-propyldimethyloctadecyl ammonium chloride (Si-QAC). Further, the step of applying an antifouling factor on the substrate may include the step of enhancing a physical factor of a surface of the substrate to prevent adhesion. The physical factor of a surface enhanced to prevent adhesion may be the smoothness of the surface. Where the surface of the substrate is a hydrogel, the physical factor enhanced to prevent adhesion may be the hydrophobic nature of the hydrogel.

In view of this disclosure, it will be seen that technologies are generally described for indwelling medical devices for detection of luminal catheter colonization. A substrate for use as an indwelling medical device may include an elevated biofilm releasing medium including a dye for release into urine on the occurrence of an elevated biofilm build-up on the substrate in the urine; and an elevated pH releasing medium including a dye on the substrate different from the dye used to detect biofilm build-up for release into urine on the occurrence of an elevated pH level in the urine. Methods of manufacture and use of the disclosed indwelling medical devices are also described.

It will be appreciated that the order of the release of the first and the second detecting material (e.g., the dye in the elevated pH releasing medium and the dye in the elevated biofilm releasing medium) is determined generally by the condition in the urine. If the biofilm forms first as is usually the case under normal conditions, then the second detecting material will be released first. If, however, the condition of an elevated pH level occurs before the biofilm formation, then the first detecting material will be released first.

This disclosure provides an indwelling medical device, illustratively a urinary catheter, that notifies the attending nurses or the patient of the catheter status by releasing dye into the output urine. The color of the dye indicates the status, i.e. if the device is colonized or if conditions conducive to encrustation are present. This disclosure improves the conditions for patients in long-term care. Additionally, the disclosure enhances aged care where urinary catheters are applied to patients who will remain catheterized for extended periods. Utilizing this disclosure may increase efficacy of antibiotic therapy by identifying catheters that are colonized and may reduce effectiveness. Additionally the may limit costly complications of catheter-associated infections.

This disclosure utilizes the fundamental processes that biofilm undergoes in order to detect its presence and allowing a sterile device to be placed. Additionally, utilizing a dual output signal—a first from the first detecting material and a second from the second detecting material—allows one device to provide information about both colonization and occlusion of the indwelling device, illustratively a catheter. The two stage response provided by this disclosure, during colonization and during crystallization, provide these and other advantages.

It will be appreciated that either or both the first and the second detecting materials may reside in the structure of the indwelling device itself, in any one or more disclosed layers, coatings or other material that may be disposed with the indwelling device, accessory components, subassemblies, or other accessory parts, or any one or more combinations thereof. The design of the material containing the second detecting material to release its detecting material payload at a different pH level that is lower than the level of pH required to release the detecting material payload containing the first detecting material makes any and other combinations possible under this disclosure.

While a range of dyes has been disclosed, it will be appreciated that it is not inclusive and in addition may include other visible dyes as well as dyes not visible to the eye. Non-visible dyes may include biocompatible fluorescing dyes with dye selection depending on the intended use. For example, where the dye needs to be viewed through tissue, dyes illustratively having a wavelength that is near or within the infrared range may provide good transmissibility for passing through tissue. In order to view dyes with an emission wave band outside of the visible spectrum of waves, energy must be delivered to the dye to excite the molecules and the resulting emission by the molecules must be collected by specialized equipment sensitive to this non-visible band of waves. Various illustrative methods and devices for delivering energy to dyes with emission outside the visible band of waves and for detecting the wavebands emitted by the dyes in response to excitation are well known. It will be appreciated that any suitable dye may be used in conjunction with the present disclosure, so long as it is effective for detection directly, such as detection through visible inspection, or indirectly, such as with the assistance of a machine or apparatus or by a test on the dye containing fluid specimen involving a chemical or other reaction, and is not unduly unsafe for the subject. Combinations of dyes may also be used with this disclosure.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A medical device for indwelling use in a urinary system, the medical device comprising:
a substrate configured to contact urine;
a first detecting material disposed with the substrate configured to be released into urine on the occurrence of an elevated pH level in the urine;
a second detecting material disposed with the substrate configured to be released into the urine on the occurrence of a biofilm on the substrate;
wherein detection of the first detecting material in the urine indicates an elevated pH level in the urine; and
wherein detection of the second detecting material in the urine indicates the presence of a biofilm on the substrate.

2. The medical device of claim 1 wherein the substrate is taken from the group of substrates consisting of the structure provided by an insertable or implantable medical device, the structure provided by an accessory associated with the insertable or implantable medical device, a coating provided to the insertable or implantable medical device or to the accessory associated with the implantable or insertable medical device, or any combination thereof.

3. The medical device of claim 1 wherein occurrence of an elevated pH level indicates a condition conducive to an occlusion of the device.

4. The medical device of claim 1 wherein the elevated pH level is at a pH level of substantially at or above about 7.4.

5. The medical device of claim 1 wherein the occurrence of an elevated biofilm on the substance indicates a condition conducive to an infection.

6. The medical device of claim 1 wherein the first detecting material is a first non-toxic dye and the second detecting material is a second non-toxic dye different from the first non-toxic dye, the first and/or second non-toxic dyes being visible in urine.

7. The medical device of claim 1 wherein the first detecting material is a first non-toxic dye and the second detecting material is a second non-toxic dye different from the first non-toxic dye, the first and second non-toxic dyes taken from the group consisting of Annatto, Chlorophyllin, Cochineal, Betanin, Curcumin, Lycopene, Yellow No. 5, β-carotene, rifampin, Yellow No. 6, tetracycline, Red No. 40, Red No. 3, Blue No. 2, Evan's Blue, Green No 3, Blue No. 1, methylene blue, indocyanine green, Riboflavin, Tartrazine, Quinoline yellow, Cochineal, Carminic acid, Azorubine, Amaranth, Cochineal Red, Erythrosine, Allura Red AC, Patent Blue V, lndigotine, Brilliant Blue FCF, Green S, Brilliant Black BN, Vegetable carbon, Capsanthian, Beta-apo-8′-carotenal, Lycopene, Ethyl ester of beta-apo-8′-carotenoic acid, Lutein, Canthaxanthin, Anthocyanins.

8. The medical device of claim 7 wherein the first dye is encapsulated in a pH responsive nanoparticle, the pH responsive nanoparticle releasing the first dye into the urine when the pH of the urine is at a pH of substantially at or above 7.4.

9. The medical device of claim 7 wherein the substrate further comprising a pH responsive hydrogel, the pH responsive hydrogel being impregnated with the second dye, the pH responsive hydrogel providing a carrier medium for the first dye, the carrier medium releasing the first dye into the urine when the pH of the urine is at a pH of substantially at or above 7.4.

10. The medical device of claim 9 wherein the coating of hydrogel comprises a carboxymethylchitosan-based hydrogel.

11. The medical device of claim 7 wherein the first dye is encapsulated using a pH responsive polyester urea, the pH responsive polyester-urea releasing the first dye into the urine at a pH level of substantially at or above 7.4.

12. The medical device of claim 7 wherein the indwelling medical device is a catheter comprising a shaft including one or more lumens which may be inserted into a subject for introduction of fluids, for removal of fluids, or both.

13. The medical device of claim 7 wherein the device is a stent.

14. The medical device of claim 12 wherein the dye is disposed along an inside wall of at least one of the one or more lumens of the catheter.

15. The medical device of claim 7 further comprising a pH reducing factor.

16. The medical device of claim 7 further comprising a calcium phyrophosphate crystal salvation factor.

17. The medical device of claim 7 wherein the substrate includes a plasticizer, the plasticizer being more responsive to metabolism of biofilm than to elevated pH levels; the second dye being incorporated into the plasticizer.

18. The medical device of claim 7 wherein the substrate includes a PVC.

19. The medical device of claim 7 further comprising antimicrobial factors.

20. The medical device of claim 19 wherein the antimicrobial factors are taken from the group of antimicrobial factors consisting of: antibacterial drugs, antiviral agents, antifungal agents, antiparasitic drugs and combinations thereof.

21. The medical device of claim 7 further comprising antifouling factors.

22. The medical device of claim 21 wherein the antifouling factors are taken from the group of antimicrobial factors consisting of antibiotics, oxidizing agents (e.g. iodine, peroxide), alcohol, bacteriocins, chelating agents, NaCl, CaCl2, MgCl2, surfactants, urea and/or antimicrobial peptides (AMPs).

23. The medical device of claim 17 wherein the substrate is embedded with a silver, copper, or the quaternary ammonium compound 3-(Trimethoxysilyl)-propyldimethyloctadecyl ammonium chloride (Si-QAC) to form an antimicrobial surface.

24. The medical device of claim 7 further comprising enzyme factors capable of damaging expolymer structure.

25. The medical device of claim 24 wherein the enzyme factors are taken from the group of enzyme factors consisting of cellulase, polysaccharide depolymerase, alginate lyase, disaggregatase, esterases, dispersin B, DNase I.

26. A process for detecting occlusion and the growth of a biofilm on an indwelling medical device comprising:
passing urine over a surface of the indwelling medical device;
degrading the surface of the indwelling medical device by a biofilm growing thereon by an amount sufficient to release a first dye, releasing a sufficient amount of the first dye from the surface of the indwelling medical device into the urine to become detectable in urine;
detecting the first dye in the urine indicating a condition conducive to an infection; degrading the surface of the indwelling medical device by an elevated pH sufficient to release a second dye therefrom; releasing a sufficient amount of the second dye from the surface of the indwelling medical device into the urine to become detectable in urine; and
detecting the second dye in the urine indicating a condition conducive to an occlusion of the indwelling device.

27. A method for making a medical device comprising:
providing a substrate for use as an indwelling medical device; applying an elevated biofilm releasing medium including a first dye to the substrate for release into urine on the occurrence of an elevated biofilm build-up on the substrate in the urine; and applying an elevated pH releasing medium including a second dye to the substrate different from the first dye for release into urine on the occurrence of an elevated pH level in the urine.

28. The method of claim 27 further comprising:
applying an antimicrobial factor on the substrate configured to be released into the urine with the release of the first dye.

29. The method of claim 28 wherein the antimicrobial factor is taken from the group of antimicrobial factors consisting of: antibacterial drugs, antiviral agents, antifungal agents, and antiparasitic drugs and combinations thereof.

30. The method of claim 27 further comprising:
applying an antifouling factor to the substrate configured to be released into the urine with the release of the first dye.

31. The method of claim 30 wherein the antifouling factor is taken from the group of antimicrobial factors consisting of antibiotics, oxidizing agents (e.g. iodine, peroxide), alcohol, bacteriocins, chelating agents, NaCl, CaCl2, MgCl2, surfactants, urea and/or antimicrobial peptides (AMPs) and combinations thereof.

32. The method of claim 30 wherein the step of applying an antifouling factor to the substrate includes the step of applying an antimicrobial surface to the substrate.

33. The method of claim 32 wherein the step of applying an antimicrobial surface to the substrate is performed by disposing with the substrate a silver, copper, or the quaternary ammonium compound 3-(Trimethoxysilyl)-propyldimethyloctadecyl ammonium chloride (Si-QAC).

34. The method of method of claim 30 wherein the step of applying an antifouling factor to the substrate includes the step of enhancing a physical factor of the substrate to prevent adhesion.

35. The method of claim 34 wherein the physical factor of the substrate is a surface enhanced to prevent adhesion is the smoothness of the surface.

36. The method of claim 27 further comprising:
applying an enzyme factor capable of damaging expolymer structure to the substrate configured to be released into the urine with the release of the second dye.

37. The method of claim 36 wherein the enzyme factor is taken from the group of enzyme factors consisting of cellulase, polysaccharide depolymerase, alginate lyase, disaggregatase, esterases, dispersin B, DNase I.

38. The method of claim 27 further comprising:
applying a pH reducing factor to the substrate configured to be released into the urine with the release of the second dye.

39. The method of claim 27 further comprising the steps of:
applying a calcium phyrophosphate crystal salvation factor to the substrate configured to be released into the urine with the release of the second dye.

40. The method of claim 39 wherein the calcium phyrophosphate crystal salvation factor is a polyphosphate.

41. The method of claim 27 wherein the occurrence of an elevated pH level indicates a condition conducive to an occlusion of the medical device.

42. The indwelling medical device of claim 27 wherein the elevated pH level is at a pH level of substantially between about 7 and 8 pH or higher.

43. The indwelling medical device of claim 27 wherein the occurrence of an elevated biofilm on the substance indicates a condition conducive to an infection.
